(19) United States
(12) Patent Application Publication
(10) Pub. No.: US 2010/0285521 A1
(43) Pub. Date: Nov. 11, 2010

(54) ADIPOSE TISSUE COLLECTION AND PRE-PROCESSING DEVICES FOR USE IN LIPOSUCTION PROCEDURE

(76) Inventors: Erik Vossman, Honolulu, HI (US); Shannon Iwami, Honolulu, HI (US); Anthony Yang, Honolulu, HI (US); Tom Cannon, Honolulu, HI (US); Hyun Joon Paek, Mililani, HI (US)

Correspondence Address: Nixon Peabody LLP, P.O. Box 60610, Palo Alto, CA, 94306, US

(21) Appl. No.: 12/437,548

(22) Filed: May 7, 2009

(51) Int. Cl. C12Q 1/04; C12M 1/00; C12N 5/02

(52) U.S. Cl. 435/34; 435/308.1; 435/379

(57) ABSTRACT

Devices and methods are described for pre-processing tissue samples containing cells of interest for various therapeutic procedures. Fibrous material may be extracted from the tissue sample through a shredding and separating device, resulting in a non-fibrous sample containing the cells of interest, which may then be washed and centrifuged to remove fluids, oil, and contaminants. The sample may then be digested in a large-volume container, and a piston within the container may push the resulting liberated cell suspension through a cell concentrator such as a hollow-fiber separation module.

### TECHNICAL FIELD

This subject matter relates generally to devices and methods for the collection and/or pre-processing of tissue, including liposuction aspirate, for use in supporting various therapeutic procedures including tissue engineering and stem cell therapies.

### BACKGROUND

Disclosed herein are methods and devices for collecting and pre-processing adipose tissue, typically aspirated during liposuction, which contain adult stem cell populations, and which can be used for various therapeutic procedures including tissue engineering and stem cell therapies.

In present practice, adipose tissue containing an adult stem cell population is typically collected from a patient during liposuction, and processed to remove the stem cells which then can be used in the therapy. Tissue can be collected in several ways, a particularly efficient one being common liposuction using a vacuum source. This lipoaspirate can be collected through an incision in the patient whereby the surgeon passes a cannula connected via tubing to the vacuum source back and forth through the appropriate cavity, releasing adipose tissue via vacuum and mechanical motion. The cannula can be a range of sizes to collect smaller or larger pieces of tissue at once. This collection procedure, however, collects more than just adipose tissue. It also collects fibrous connective tissues resident in the adipose deposits, and it also collects fluids such as blood, oil, and tumescence fluid, which are generally considered to be undesirable.

Typically, after the lipoaspirate is collected, it will be processed by a technician, usually in a sterile flow hood or sterile environment. If the tissue has been collected with a large-bore cannula, the tissue is generally chopped into smaller pieces for ease in processing. This allows for a more efficient release of desired cells from the tissue and allows the later-used enzyme to break down the tissue more efficiently. This chopping step may be eliminated through the use of a smaller bore cannula; however, the smaller the cannula, the longer time the surgeon must take in collecting the tissue, and there may be medical, efficiency, or other reasons to minimize the collection time.

When the collected tissue is of sufficient size, it is typically placed in a small-pore sieve, and a sterile wash buffer or fluid such as saline or phosphate buffered saline (PBS) is added. As the wash buffer is added, a technician will typically push the tissue back and forth with a cell scraper against the sieve screen aseptically. This allows the wash buffer to clean the tissue while also allowing the fluid to drain through the sieve into a beaker. Eventually, this back and forth motion and the adding of wash buffer results in a relatively concentrated tissue sample, relatively free of blood, oil, tumescent fluid, and other undesirable components.

Alternatively to the above processing method, aspirated adipose tissue can be washed and concentrated inline while the surgeon is collecting tissue. For example, this can be done using a sterile inline filter such as the Genesis Biosystems, Inc. Lipivage™ fat harvester system. The surgeon can then aspirate wash buffer or saline through the same cannula to help wash and clean the tissue sample. However, this method has the disadvantage that the surgeon can only collect roughly 10 to 20 cubic centimeters of tissue at a time. Also, the system does not remove fibrous connective tissue from the sample. Another flaw with the method is that it may be difficult for some surgeons to use. In particular, existing inline filter systems may be cumbersome to handle, and may break during the procedure. There may also be higher costs associated with the collection procedure because a surgical team must purchase the inline filter device.

As an alternative to an inline filter, the tissue may be collected by using a strainer or filter basket within the vacuum canister. For example, the surgeon may use a Disposable Specimen Cup provided by Medela Healthcare U.S., which is a filter basket for use in the company's disposable or non-disposable vacuum canister. However, this has the same disadvantages as an inline filter, including the limitation that it does not remove fibrous connective tissue. In addition, a system such as this can have problems with non-uniform washing and concentrating of tissue. In addition, unless the vacuum canister and specimen collection system is properly sterilized and maintainably sterile, the sterile field must be broken to retrieve the sample into a syringe that can transfer the sample to another processing device.

In addition to removing undesirable components such as oil and blood, fibrous tissue is typically removed as well. This may be done manually during the cleaning and concentrating steps described above. A technician may do this by recognizing the difference in color of adipose and fibrous tissue, using one of various tools to manually pull this tissue from the sample and discard it. However, this procedure takes considerable time and effort, and requires the user to be trained in the method, and in distinguishing fibrous tissue components from adipose tissue. The overall process described above can require about one hour to complete by at least one trained technician using multiple tools within a sterile environment.

The sample is then typically digested in what is typically a manual process which involves the addition of a digestion enzyme such as collagenase, which produces a cell suspension.

The present disclosure provides methods and devices which address the shortcomings of the prior art, thus improving the collection and pre-processing of tissue by eliminating the need for user intervention, reducing the likelihood of compromising the sterile barrier, and reducing the processing time, as well as other benefits and uses as disclosed herein.

### BRIEF SUMMARY

The present disclosure relates to methods and devices for collecting and pre-processing adipose tissue, typically aspirated during liposuction, which contain adult stem cell populations, and which can be used for various therapeutic procedures including tissue engineering and stem cell therapies. Various embodiments are possible, a number of which are exemplified here.

In one embodiment of the present disclosure, there is provided a tissue processing device for extracting cells of interest from an input composition comprising a tissue sample composition and one or more liquid phase compositions, comprising: first automated means for shredding at least a portion of the input composition, and for separating said portion of the input composition into a fibrous composition and an intermediate cell product composition, wherein the fibrous composition is relatively rich in a fibrous tissue component and the intermediate cell product composition is relatively rich in the cells of interest; second automated means for separating the one or more liquid phase compositions from the fibrous composition or the intermediate cell product composition, or both; and an electronic control system capable of starting and stopping the operation of the first automated separating means and for starting and stopping the second automated separating means.

In another embodiment, there is provided a method for processing an input composition comprising a tissue sample composition and one or more liquid phase compositions to extract cells of interest, comprising the steps of: engaging a first automated apparatus for: shredding at least a portion of the input composition; and separating said portion of the input composition into a fibrous composition that is relatively rich in a fibrous tissue component and an intermediate cell product composition that is relatively rich in the cells of interest; and engaging a second automated apparatus to separate the one or more liquid phases from the fibrous composition or the intermediate cell product composition, or both.

In another embodiment, there is provided a tissue processing device for extracting cells of interest from a first input composition comprising a tissue sample composition and one or more liquid phase compositions, comprising: a container comprising a first compartment and a second compartment; first separation means within the first compartment for separating at least a portion of the input composition into a first solid composition that is relatively rich in the tissue sample composition, and a first liquid composition that is relatively rich in the one or more liquid phase compositions; a gap between the first compartment and the second compartment; means within the gap for shredding at least a portion of the first solid composition, and for separating said portion of the first solid composition into a fibrous composition and an intermediate cell product composition so as to transfer at least a portion of the intermediate cell product composition to the second compartment while retaining the majority of the fibrous composition outside the second compartment, wherein the fibrous composition is relatively rich in a fibrous tissue component and the intermediate cell product composition is relatively rich in the cells of interest.

In another embodiment, there is provided a method for processing an input composition comprising a tissue sample composition and one or more liquid phase compositions to extract cells of interest, comprising the steps of: injecting the input composition into the first compartment of a container comprising: a first compartment and a second compartment; a gap between the first compartment and the second compartment; and a first plurality of blades within the gap; separating at least a portion of the input composition into a first solid composition that is relatively rich in the tissue sample composition, and a first liquid composition that is relatively rich in the one or more liquid phase compositions; sweeping the first plurality of blades against a second plurality of blades in contact with the portion of the first solid composition, wherein the second plurality of blades is configured to be in an offset tooth pattern with the first plurality of blades, and wherein the openings defined by the spaces between the offset teeth are large enough to permit passage of at least a portion of the first solid composition, and small enough to prevent passage of the majority of the fibrous composition after the first plurality of blades have swept against the second plurality of blades; and separating said portion of the first solid composition into a fibrous composition and an intermediate cell product composition so as to transfer at least a portion of the intermediate cell product composition to the second compartment while retaining the majority of the fibrous composition outside the second compartment, wherein the fibrous composition is relatively rich in a fibrous tissue component and the intermediate cell product composition is relatively rich in the cells of interest.

In another embodiment, there is provided a tissue processing device for extracting cells of interest from a tissue sample composition, comprising: a digestion chamber comprising a first section and a second section, the first section and the second section separated by a first filter, wherein the first filter is configured to allow passage of particles of a particular size or smaller, while retaining particles of the particular size or larger within the first section of the digestion chamber, wherein the particular size is larger than the cells of interest; agitation means for agitating a composition within the digestion chamber; a piston configured within the first section so as to be capable of displacing fluids in either of two directions across the first filter; and a system of one or more valves configurable in a first configuration to allow fluid communication between the second section and a waste container, configurable in a second configuration to allow fluid communication between the second section and a suspension medium container, and configurable in a third configuration to allow fluid communication with the inlet of a cell concentrator.

In another embodiment, there is provided a method for processing a tissue sample composition to extract cells of interest, comprising the steps of: introducing the tissue sample composition into a digestion chamber comprising a first section and a second section, the first section and the second section separated by a first filter, wherein the first filter is configured to allow passage of particles of a particular size or smaller, while retaining particles of the particular size or larger within the first section of the digestion chamber, wherein the particular size is larger than the cells of interest; introducing a suspension medium into the first section of the digestion chamber; allowing the contents of the first section of the digestion chamber to digest for a predetermined length of time, to create a digested cell composition; pushing or suctioning at least a portion of the digested cell composition through the first filter; and passing said portion of the digested cell composition through a passage toward the inlet of a cell concentrator; passing at least a part of said portion of the digested cell composition through the cell concentrator.

### BRIEF DESCRIPTION OF THE DRAWINGS

The accompanying drawings, which are incorporated into and constitute a part of this specification, illustrate one or more exemplary embodiments of the inventions disclosed herein and, together with the detailed description, serve to explain the principles and exemplary implementations of these inventions. One of skill in the art will understand that the drawings are illustrative only, and that what is depicted therein may be adapted based on the text of the specification or the common knowledge within the relevant field of art.

In the drawings:

FIG. 1a shows an embodiment of a device for shredding lipoaspirate to remove fibrous tissue and undesirable fluids, and to collect adipose cells of interest. FIG. 1b shows an embodiment of interlocking combs or teeth.

FIG. 2a shows another embodiment of a device for shredding and separating adipose cells from fibrous tissue and undesirable fluids in one orientation. FIG. 2b shows the same device in a rotated orientation.

FIG. 3 shows an apparatus for digesting and filtering adipose tissue, and concentrating adipose cells.

### DETAILED DESCRIPTION

Various example embodiments of the present inventions are described herein in the context of devices and methods for the collection and/or pre-processing of tissue, including liposuction aspirate, for use in supporting various therapeutic procedures including tissue engineering and stem cell therapies. Example embodiments are disclosed, including methods, devices, and systems. Such embodiments have the advantage that they improve the collection and pre-processing of tissue by, among other things, eliminating or reducing the need for user intervention, reducing the amount of time that the sterile barrier is compromised, and improving the quality of tissue for processing and obtaining regenerative cells using a process such as the Tissue Genesis, Inc. Cell Isolation System. These embodiments can also improve the quality of reinjectable tissue by eliminating the fibrous tissue from adipose samples and cleaning the tissue of blood, tumescent fluid, oils, and other undesirable fluids.

Those of ordinary skill in the art will understand that the following detailed description is illustrative only and is not intended to be in any way limiting. Other embodiments of the present inventions will readily suggest themselves to such skilled persons having the benefit of this disclosure, in light of what is known in the relevant arts, such as the arts of cell therapy, tissue engineering, and other related areas. Reference will now be made in detail to exemplary implementations of the present inventions as illustrated in the accompanying drawings.

In the interest of clarity, not all of the routine features of the exemplary implementations described herein are shown and described. It will of course be appreciated that in the development of any such actual implementation, numerous implementation-specific decisions must be made in order to achieve the specific goals of the developer, such as compliance with regulatory, safety, social, ethical, and business-related constraints, and that these specific goals will vary from one implementation to another and from one developer to another. Moreover, it will be appreciated that such a developmental effort might be complex and time-consuming, but would nevertheless be a routine undertaking of engineering and development for those of ordinary skill in the art having the benefit of this disclosure.

Throughout the present disclosure, relevant terms are to be understood consistently with their typical meanings established in the relevant art. However, without limiting the scope of the present disclosure, further clarifications and descriptions are provided for relevant terms and concepts as set forth below:

The term automated is used herein according to its normal and ordinary meaning. In particular, once an automated device is set up, activated, or engaged to perform its function, it can perform that function without further direct manual intervention until the function is complete. For example, a computer or microprocessor may be programmed to perform the function, which is then carried out automatically. In another example, a user may press a button, pull a lever, of flip a switch to cause a function or group of functions to be performed. In another example, a user may activate multiple buttons, levers, keys, or switches, in a particular sequence, or simultaneously, to perform a function or functions.

The term fibrous tissue or fibrous tissue component as used herein has its normal and customary meaning in the medical field, which includes a tissue or tissue component comprising fibers such as, without limitation, collagen fibers, keratin fibers, elastin fibers, other protein fibers, or other fibers found in tissue. Many biological tissues may have a fibrous tissue component. For example, adipose tissue may comprise both fibrous tissue components (such as collagen fibers) and non-fibrous tissue components (such as adipocytes, fibroblasts, macrophages, endothelial cells, and adipose stromal cells (ASCs)).

The term filter as used herein has its normal and customary meaning in the field of engineering, and additionally includes, among other things, a device or series of devices that retain some components of a mixture while allowing others to pass through. In some cases, the selection of what passes through the filter is based on size; for example, a filter retains larger molecules or particles while allowing smaller ones to pass through. Examples of simple size-based filters include a mesh or screen, a sieve, or a porous membrane, which may come in many varieties and compositions known in the art. For example, without limitation, a filter may comprise a wire or polymer screen; woven, aligned, or tangled fibers; a plate or membrane with holes, channels, or pores; a bed of loose or adherent particles, such as a powder, a series of porous hollow fibers, or other types of filters known in the art. A filter may also include multiple such components or stages; for example, a filter may comprise a series of parallel membranes or parallel porous hollow fibers, or filters operating in a series or parallel configuration. Each of these filters, and others, may be suitable for separating cells or pieces of tissue from blood, oils, tumescent fluids, and wash fluids. For example, a filter may be used that has smaller openings or channels than the cells or pieces of tissue, but the openings are larger than the blood, oils, and tumescent fluids, thus allowing them to pass through. In addition, among other things, meshes or screens may be useful to separate cells from fibrous tissue, or cells from other cells, or certain types of tissue from other types of tissue.

The term lipoaspirate refers to material obtained by aspiration during surgical procedures such as liposuction. When reference is made herein to lipoaspirate, one of skill in the art will understand that such material may be obtained in a number of ways, including liposuction or other methods. As an alternative to lipoaspirate, the disclosures herein may be carried out through equivalent means such as the use of lipectomy specimens which may, for example, be extracted surgically with a scalpel.

The term patient is used herein according to its normal and ordinary meaning, and in particular refers to the subject of any treatment, therapy, or surgical procedure, or any experimental or investigational study as to the viability of any treatment, therapy, or surgical procedure. A patient may include a human or a non-human animal. Status as a patient does not necessarily require the existence of a physician or health care provider; for example, a patient may self-administer a treatment or therapy. For purposes of this disclosure, the term patient may also include a cadaver or dead animal, so long as cells of interest in the patient remain viable.

The term piston is used herein according to its normal and ordinary meaning, and in particular refers to a mechanical component that fits within a container and is capable, by its motion of deformation, of displacing or being displaced by a fluid. Non-limiting examples of pistons include plungers (such as those found within syringes), or diaphragms. The term piston may include, and is equivalent to, a series of pistons which may operate cooperatively or in coordination.

The present disclosure describes a number of embodiments, including a pre-processing unit with the function of automatically removing fibrous tissue from a tissue sample from a patient, washing and cleaning the tissue sample, isolating cells of interest, and concentrating them without user intervention. All such functions may, in one preferred embodiment, be incorporated into a single disposable vacuum canister that can be placed inline during a liposuction procedure similar to a vacuum waste canister.

In one example, a device may use a mechanical action or filter to collect fibrous tissue while allowing the desirable adipose tissue to pass through. This could be done, in one embodiment, by using a mechanical comb or rotating comb device. The device might also be configured to concentrate and wash the desirable adipose tissue by, in one embodiment, using a filter and preferably a basket-like catch device within a vacuum canister. A preferred embodiment would be one that mimics the user interaction between a manual cell scraper and sieve used known in the prior art. For example, a filtered centrifuge may incorporate a method to comb, scrape, or remove the tissue from a filter or screen and then re-centrifuge. This could be done multiple times and all be done during the collection and washing process. Any excess blood, tumescent, oil, and wash fluid may be collected in a vacuum canister to be discharged or used for other purposes.

In addition, a collection system may be used for easy retrieval of the cells of interest via, in one embodiment, a syringe and Luer connection. Thus, a system may be provided for directing or scraping the tissue off of the filter, screen, or centrifuge filter/screen, towards an easy collection chamber. The chamber would then have a port to connect a syringe and collect the tissue. This could be done, in one embodiment, by engaging a scraper that directs the cleaned concentrated tissue up towards a port which fills a connected syringe automatically. A user could remove the syringe once full and connect another syringe to collect the remaining tissue. Alternatively, the port could directly connect to an apparatus for further processing.

A preferred embodiment would be a fully automated device run off of just the vacuum air of the aspirator commonly used for liposuction and to be able to be run continuously. Alternatively, the device could run off of a power supply with separate pumps and motors.

In another embodiment, the user may collect the tissue into the canister and activate either a washing, concentrating, or fibrous tissue removing step in succession by manually switching or engaging the correct mechanisms on the vacuum canister. These switches could have indications or them to let one know which to engage next. Alternatively, the entire process could be performed by automatic control by a microprocessor or computer.

In another embodiment, the device may include a collection and transfer vessel inside a vacuum canister to be removed in an aseptic manner and allow for manipulation away from the patient and vacuum canister. This collection and transfer vessel could then have connections to integrate into another fluidics system or another apparatus for further processing.

In an another embodiment, the functions of suction, collection, processing for fibrous tissue removal, washing, and concentrating, may be performed as separate steps on a batch of tissue sample, and repeated as necessary. For instance, an embodiment may contain a fibrous tissue removal tool and a wash mechanism that continuously processes the same tissue. This could be advantageous for highly fibrous tissue to ensure proper removal of said tissue.

Another embodiment could place the system to remove connective tissue in-line in the tubing connected between a surgical cannula and a vacuum canister. The vacuum canister could then contain the washing and concentrating device.

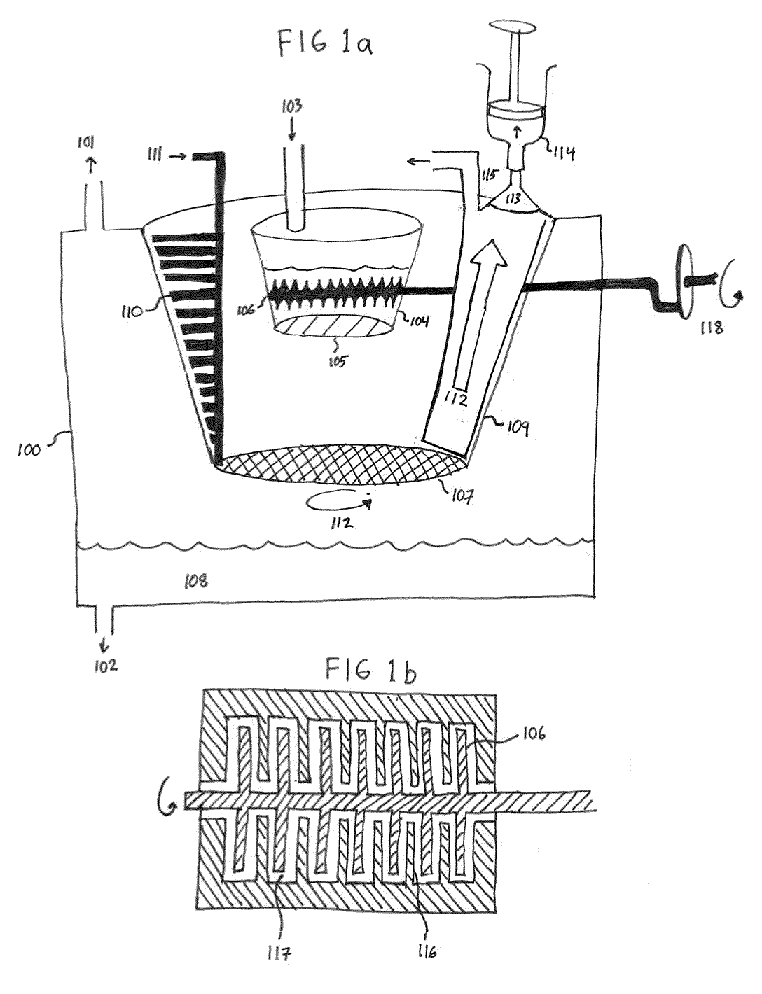

An example embodiment is shown in part in FIG. 1a, a tissue processing device is provided for extracting cells of interest from a tissue sample. This device may include an external container 100 connected to a vacuum pump via vapor outlet 101. Container 100 may also contain a liquid waste outlet 102 for draining liquid waste via a vacuum pump, syringe, gravity, or other equivalent means, and may include a valve. In one embodiment, container 100 may be treated disposable, and all wastes 108 may be left inside for later disposal.

The device may contain a tissue inlet 103 which in one embodiment may be connected to a surgical catheter for use in a liposuction procedure. Surgical methods of liposuction are known in the art. Inlet 103 may be connected to a combing or shredding/separating device which may contain a mechanical process for shredding the tissue sample and removing from within it the fibrous tissue. This combing or shredding device may contain a solid surface comprising a plurality of perforations large enough to permit passage of at least a portion of the intermediate cell product composition, and small enough to retain the majority of the fibrous composition. In one embodiment, the shredding device may consist of a basket 104, part of which might, in one embodiment, comprise an optional screen 105 which is narrow enough to retain fibrous tissue components, but wide enough to admit an intermediate cell product composition and/or any fluids such as wash fluid or tumescent fluids. The intermediate cell product composition may include tissue with most or all of the fibrous component removed. In one embodiment, basket 104 may include a set of blades 106, which in one embodiment could be a rod or cylinder with protruding blades or combs. These blades or combs may rotate or otherwise move in contact with or near a surface which may in one embodiment include a filter. In a preferred embodiment, shown in FIG. 1b, the blades 106 may include two or more sets of interlocking combs or teeth, where at least one set of combs 116 is stationary and another set of combs 106 may rotate past the first set of combs, thus shredding the tissue sample caught between the combs. The space between the combs 117 may act as a filter to prevent fibrous tissue from passing through the barrier created by the combs. In addition, fibrous tissue may become wound around the combs and thereby be prevented from passing through the barrier. This configuration may be, in one embodiment, similar to that of an herb mill.

In an alternate embodiment, the shredding device may comprise a solid surface containing a plurality of perforations large enough to permit passage of at least a portion of the cells of interest, as well as liquids, and small enough to retain the majority of the fibrous composition. A piston may be configured to press the tissue sample against the solid surface, to extrude the cells of interest while retaining the fibrous tissue. This device may take a form similar to a potato ricer.

In another embodiment, the shredding device may comprise a set of blades configured to scrape against a surface, such as the surface of a mesh or screen, while the blades and the surface are in contact with the tissue sample.

The processes above may be capable of removing the majority of the fibrous tissue from a sample. Preferably, 90% of the fibrous tissue may be removed; and most preferably, 100% of the fibrous tissue may be removed according to the above disclosure.

The shredding device may be rotated or otherwise operated by an automated motor 118 which may receive signals directing it to begin the mechanical operation of the shredding device by causing rotation of the blades discussed above, causing the movement of the piston described above, or otherwise causing linear or rotary movement of a set of blades or a piston relative to a surface containing openings sufficient to allow passage of the cells of interest.

The device may include screens and filters, such as screens 105 and 107, which may allow any liberated fluid 108, including wash fluid, blood, tumescent fluids, or oils, to collect, typically for disposal as waste, such as via waste outlet 102. During the shredding and separation process, the sample may be washed or cleaned with a wash buffer such as saline, PBS, or other equivalent fluids known in the art. This fluid may be collected as waste 108 and optionally disposed into a waste stream 102.

The automated device may also contain a second separating function for separating the liberated cell product composition from any excess fluids or contaminants. In one embodiment, this may be accomplished through the use of a second basket or container 109, which may contain holes, openings, or perforations large enough to permit passage of at least a portion of the intermediate cell product composition, and small enough to retain the majority of the fibrous composition. Container 109 may include one or more sets of blades 110 configured to sweep against the side of the basket 109. Alternatively, the blades may sweep against another set of interlocking or offset blades.

Prior to or during the second separation function, the basket 109 may be irrigated by a wash or cleaning fluid, which may, in one embodiment, be pumped through the combs via inlet 111, out the end of each comb blade. This may serve to directly clean the basket surface 109, which otherwise may become clogged with particle matter.

The basket 109 may be configured to rotate (see 112), thus creating a centrifugal force against the walls of the basket 109, which may cause one or more liquid phases in the basket (including biological fluids from the tissue sample and wash or cleaning fluids) to migrate to the basket wall 109 and pass through the basket filter into the waste pool 108. A motor may be provided to cause rotation of the basket 109, which may be controlled by a switch or microprocessor so that the process is automated and does not require direct manual intervention.

The second separation function may proceed in repeated stages, wherein a wash or cleaning fluid is introduced into the basket 109, or particularly against the walls of the basket, followed by centrifugation.

In an alternative embodiment to the use of combing blades 110, the basket 109 may contain agitation means, such as stirring blades, stirring bars, impellers, or other agitation means for agitating the intermediate cell product composition within the basket to ensure that it is penetrated and thoroughly washed with wash buffer, and to ensure that the second separation function is accomplished by allowing fluids to migrate to the basket wall 109.

In one embodiment, the basket wall 109 may include a mesh or fabric made of wire, polymer, or other equivalent materials. In another preferred embodiment, the basket wall may contain a large number of embedded beads, where each bead is capable of rotating around an axis. For example, beads may be strung along the wire of a wire mesh, and be free to rotate around the wire, or woven into a fabric, where each bead is free to rotate around a thread of the fabric. Freely-rotating beads can aid in allowing fluids to pass through the mesh, but may help to prevent tissue from clogging the openings, because the tissue may slide off or fail to stick to the beads.

The basket may be provided with a system for collecting the intermediate cell product composition for further use or processing. This may be accomplished, in one embodiment, by a blade 112 or series of blades or vanes which direct the washed and concentrated cell composition toward a cavity 113, where the composition may be extracted, such as by a syringe 114, a bag or container, or by a conduit to another instrument for further processing. The less dense parts of the composition directed by these blades or vanes may be routed via channel 115, either to recycle the material back to baskets 104 or 109, to further processing, or to a waste stream.

A device similar to that shown in FIG. 1a or as described above may be used in many ways. In one embodiment, an input composition comprising the tissue sample may be introduced into container 104, and the device may be configured or instructed to shred the input composition and to separate the fibrous composition from the cell-rich non-fibrous composition as described above. The cell-rich composition may then be introduced into a basket 109, and then an operator or computer may instruct the system to spin the basket 109 to create a centrifugal force to drain fluids across a portion of the basket which serves as a filter.

Before, during, and after, centrifugation, wash fluids may be added to the basket through, for example, a special inlet to the basket, or an inlet 111 to the combs, or through the surgical inlet 103. After washing, the centrifugation step may be repeated any number of times, until the cell-rich composition in the basket is sufficiently clean and concentrated for the intended use. At that point, the cell-rich composition may be extracted from the basket manually, by a scoop, or through a drain, etc. In one embodiment, the cell-rich composition may be directed by a blade 112 or series of blades into a port 113. Part of this composition, may be directed into a bypass 115 as described above. The contents of the port may be removed by syringe via a port such as a Luer lock, or may be pushed or suctioned through a channel to another device after the opening of a valve.

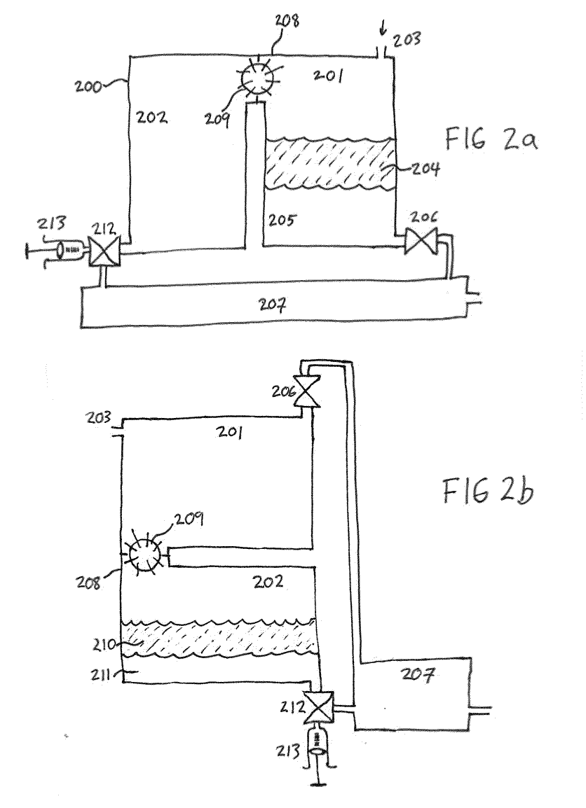

In another embodiment of the present disclosure, shown in part in FIG. 2a and FIG. 2b, a tissue processing device may be provided which includes a container 200 with two separate compartments 201 and 202. Tissue specimens from a patient may be introduced into compartment 201 via an inlet 203, which may in one embodiment be connected to a cannula during liposuction. The force of gravity, which may in an alternative embodiment be enhanced by centrifugation by rotating the container 200 and/or compartment 201 about an axis, may cause the contents of compartment 201 to separate into two or more phases or factions 204 and 205, which may for example comprise two liquid phases, or a solid an liquid phase. In one embodiment, where the tissue sample comprises lipoaspirate, faction 204 may comprise fatty tissue, while 205 may comprise tumescent fluids, blood, oil, or wash fluids. Before further processing, faction 205 may be drained, suctioned, or pushed via valve 206 into a waste container 207. Optionally, a sensor may be configured in the compartment or in the passage toward container 207 which senses the composition of the drained fluids, or of the contents of compartment 201, and stops the drainage before faction 204 is expelled into waste 207.

After compartment 201 is drained, the contents of faction 204 may be introduced into gap 208, which contains a shredding apparatus 209. The shredding apparatus may, in one embodiment, contain offset sets of blades or combs similar to that depicted in FIG. 1b, or equivalent shredding devices, or other configurations of blades moving past each other while preventing the passage of fibrous tissue.

In one embodiment, the introduction of faction 204 into gap 208 may be aided by rotation of compartment 200, for example into the configuration shown in FIG. 2b. Alternatively, compartment 201 may be separately movable from compartment 202 and one or both of them manipulated so as to introduce faction 204 into the gap 208. In another embodiment, the gap 208 may itself be moveable, while compartments 201 and/or 202 remain stationary, which might be possible, for example, if container 200 is made of a flexible material.

After shredding and separation, the fibrous components of faction 204 may remain in compartment 201 or remain wrapped around the shredding apparatus 209. The resulting composition which makes its way into compartment 202 may preferably be free of fibrous tissue. In less preferred embodiments, the percentage of total fibrous tissue making its way into compartment 202 may be less than 10%, or less than 50%.

The force of gravity, which may in an alternative embodiment be enhanced by centrifugation by rotating the container 200 and/or compartment 202 about an axis, may cause the contents of compartment 202 to separate into two factions or phases 210 and 211, which may for example comprise two liquid phases, or a solid and liquid phase. In one embodiment, faction 210 may comprise solid tissue, including the cells of interest, while 211 may comprise tumescent fluids, blood, oil, or wash fluids. In one embodiment, one of the factions 210 or 211 may comprise solid tissue, such as the cells of interest, leftover fibrous tissue, or solid waste, while the other faction may comprise tumescent fluids, blood, oil, or wash fluids. In another embodiment, faction 210 may comprise a liquid oil phase, and faction 211 may comprise a liquid aqueous phase.

In one embodiment, if faction 211 is a waste phase, it may be drained, suctioned, or pushed via valve 212, which may be a three-way valve, into waste container 207. Optionally, a sensor may be configured in compartment 202, in valve 212, or in the passage toward container 207 which senses the composition of the drained fluids, or of the contents of compartment 202, and stops the drainage before a significant amount of faction 210 is expelled into waste 207. Then, faction 210 may be removed from compartment 202 by, in one embodiment, syringe 213, or by alternative means such as a vacuum trap, a bag, or a conduit to another device for further processing.

In an alternate embodiment, if faction 210 is a waste phase, faction 211 may be removed from compartment 202 by draining, suction, or pushing via valve 212 into, in one embodiment, syringe 213, or into alternative means such as a vacuum trap, a bag, or a conduit to another device for further processing. Optionally, a sensor may be configured in compartment 202, in valve 212, or in any component (such as syringe 213) downstream from valve 212, which senses the composition of the drained fluids, or of the contents of compartment 202, and stops the drainage before the collected composition is contaminated by waste faction 210. Then, faction 210 may be drained, suctioned, or pushed via valve 212 into waste container 207.

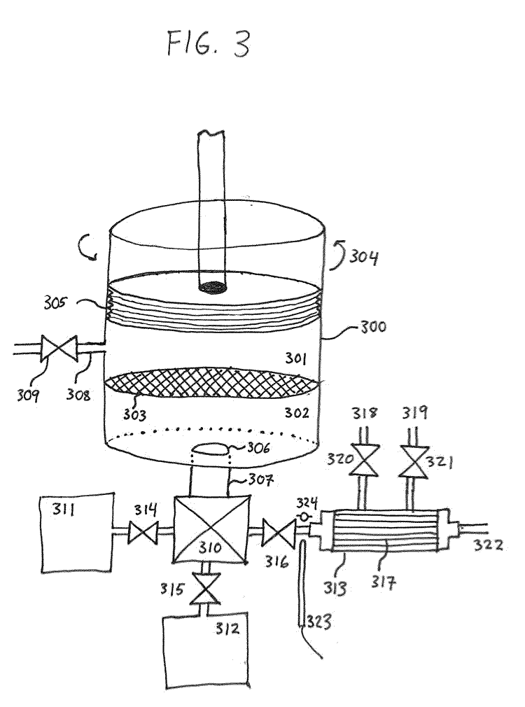

In another embodiment of the present disclosure, shown in part in FIG. 3, a tissue processing chamber may be constructed and used for extracting cells of interest from a tissue sample composition. This device may comprise a digestion chamber 300 which may include two sections 301 and 302. These two sections may be separated by a filter 303, which can allow passage of particles the size of the cells of interest, while retaining larger particles within section 301. The filter 303 may, for example, be a mesh attached securely to the walls of the digestion chamber 300. The digestion chamber 300 may be a cylinder, but also may be any other shape, including for example a right prism. The filter 303 may be located anywhere within the chamber 300, including at outlet 306, in which case section 302 will comprise passage 307. The chamber 300 may also include an inlet 308 to section 301 which may preferably be regulated by a valve 309. Inlet 308 can allow for the input of fat or other tissue sample into section 301. Inlet 308 can also allow for removal of the contents of chamber 300. Valve 309 should preferably be capable of being fully open or fully closed, and should be of sufficient diameter to allow passage of the fat or tissue sample of interest. This sample may originate from a number of sources, including for example via a direct connection from a raw tissue processing device such as those shown in FIG. 1a and FIG. 2a. The sample may also originate from a directly-connected cannula for use during a liposuction process, or the sample may be manually inserted through inlet 308 by a technician.

The digestion chamber 300 may include agitation means 304 for agitating a composition within the digestion chamber. The agitation means may include, for example, a motor or motors which cause the digestion chamber 300 to rotate about an axis. Alternatively, the motor may cause the digestion chamber 300 to rotate in one direction about an axis, then stop rotating, then rotate in the opposite direction about the same axis, which may repeat multiple times. Alternatively, a motor may cause the digestion chamber 300 to shake back and forth in some direction. The digestion chamber may also, for example, contain a stir bar such as a magnetic stir bar, or an impeller or other configuration of blades that may rotate or move linearly back and forth to agitate the contents of the digestion chamber 300. Agitation may also be accomplished by a vibrating device, or by movement of piston 305. One of skill in the art will also recognize other equivalent means for agitation.

The section 301 may include a piston 305 which can displace fluids in either of two directions across the filter 303. This piston may form a movable, aseptic seal with an internal wall of the digestion chamber 300. Alternatively, the piston may for example be a deformable diaphragm fixedly attached to the digestion chamber 300, capable of displacing fluids by deforming its shape.

A valve or series of one or more valves 310 may be connected to section 302 via passage 307. Valve 310 may comprise a single four-way valve, or may comprise a series of interconnected valves with connections to any of three components 310, 312, and 313. Valve 310 may be operated in a plurality of modes, including a mode which allows fluid connection between section 302 and a waste container 311, a mode which allows fluid connection between section 302 and a suspension medium container 312, and a mode which allows fluid connection between section 302 and a cell concentrator 313, via an optional valve 316, which is preferably a one-way valve allowing passage in the direction of cell concentrator 313. Valve 310 may optionally comprise a series of connected valves, which may also contain various intermediate components or chambers.

Waste container 311 may preferably be a bag or other container suitable for disposal of biological waste, such as by incineration. The connection between valve 310 and waste container 311 may comprise an optional valve 314, preferably a one-way valve to prevent back-flow out of waste container 311. Waste container 311 may also optionally comprise a syringe for suction into the container 311. Similarly, suspension medium container 312 may connect to valve 310 via an optional valve 315, preferably a one-way valve, and may be connected via a removable connection. Container 312 may preferably be configured so as not to become contaminated with biological tissues, so that it may be disposed as non-biological waste, or refilled or recycled.

Inside the cell concentrator 313 or in or near the passageway or inside any intermediate component from valve 310 to the cell concentrator 313, there may be a sensor system for measuring some property related to the composition of a cell suspension or other liquid moving through the passageway. For example, the sensor may comprise, without limitation, a calorimeter, a photometer, a spectrophotometer, a fluorimeter detector, an ohmmeter, an ion-selective electrode, a refractometer, a thermal conductivity meter, a densitometer, a reflectometer, a meter for measuring the speed of sound in the liquid, or any other sensor which measures a physical or chemical property of the fluid stream, of which there are many known by one of skill in the art, all of which are to be thought of as equivalent for this purpose. FIG. 3 shows one example sensor which includes an optical fiber 323 on one side of the stream and a photodiode 324 on the opposite side covered by a filter, the filter selected to best distinguish between fatty oil and a suspension of adipose cells. If the contents of the digestion chamber 300 are being pushed by the plunger 305 to the cell concentrator 313, a control system may be configured to monitor a sensor signal from the sensor, and stop the plunger 305 when there is an abrupt change in the sensor signal indicating a change in the composition of the stream entering the cell concentrator 313. In one example, such a change may occur upon the passage of a phase transition between a suspension of adipose cells and a less dense fatty oil phase.

The cell concentrator 313 may take a number of forms, many of which are known in the art; for example, cells may be concentrated through sedimentation from a suspension such as by gravity or centrifugation (including counter-streaming centrifugation), and the cell-depleted fluid may be drained or aspirated. Cells may also be concentrated by placing the cell suspension on one side of a filter and applying a vacuum to the other side to remove excess fluid. Preferably, the cell concentrator may include a chamber separated into two sections by a filter which will admit liquids but not the cells of interest. According to this embodiment, the first section is designed to contain the cells of interest, and the section is designed to contain a cleaning or rinsing solution which may cross the membrane into the first section, as well as excess fluids which cross the membrane from the first section to the second section, thus concentrating the cell suspension. Shown in FIG. 3 is a hollow-fiber filtration device 313 in which the cells are preferably passed through the hollow fibers 317. On the side of the hollow fibers opposite the cells, a cleaning or rinsing solution such as, for example, saline or phosphate buffered saline, may be introduced to device 313 and/or removed from device 313 through inlets 318 and/or 319, which may be regulated by optional valves 320 and 321, respectively.

The cleaning or rinsing solution may, in one embodiment, be circulated into one of inlets 318 or 319, and removed from the other, which may result in diffusion of extracellular fluids or enzymes into the circulating fluid and out of the cell suspension. Inlets 318 and/or 319 may also be used to remove excess fluids from the cell suspension, and may be connected to a device, such as a pump or syringe, for producing a vacuum to draw the excess fluids, including any cleaning or rinsing solutions, across the hollow fiber membranes 317.

After concentration in the cell concentrator, cells may be removed through outlet 322 and/or routed to a cell culturing system. Removal may be facilitated by flushing the fibers with a small amount of suspension medium, which may in one embodiment be introduced through a valve near the input of cell concentrator 313.

Preferably, the cell concentrator 313 will have a small amount of dead space between the opening of the concentrator 313 and the beginning of capillary fibers 317. This dead space can trap any residual undigested connective tissue. Preferably, if adipose stromal cells (ASCs) are the cells of interest, the hollow fibers 317 will be slightly larger than the ASCs.

A tissue processing device such as that shown in FIG. 3 may be used in many ways, including the following: a tissue sample composition from a patient may be processed according to the present disclosure using devices such as those shown in FIG. 1a and FIG. 2a, and may then be inserted into section 301 of the digestion chamber 300. Alternatively, an unprocessed tissue sample, such as lipoaspirate, may be directly inserted into section 301. The tissue sample may contain tumescent fluids, which may be allowed to drain through filter 303. In one embodiment, wash buffer fluid may be added to section 301 of chamber 300, and mixed by agitation, preferably by rotation (see 304) of the chamber 300. Then the wash buffer fluid may be drained across the filter 303, by for example gravity, suction, by pressure from piston 305, or other means. Waste from chamber 300 may be routed via valve system 310 to waste container or bag 311.

A suspension medium may be added to section 301 of the chamber 300, preferably from suspension medium container 312, and may be introduced by gravity, pressure from for example a syringe, or preferably, from suction produced by piston 305, which may be configured to draw the suspension medium into chamber section 301 via valve system 310. A suitable suspension medium may comprise Adipase™, supplied by Tissue Genesis, Inc. The suspension medium may also comprise other enzymes or chemicals known in the art which are useful for digesting adipose tissue, such as without limitation collagenase. If collagenase is used, the medium may also comprise M199, M199E, PBS, saline, or di-cation free DPBS, for example. Digestion of adipose tissue preferably takes place at about 37° C., and may proceed for a time sufficient to digest the tissue sample, which may typically be 20 minutes, 30 minutes, an hour, or two hours. Optionally, after digestion has proceeded sufficiently, a composition (such as, without limitation, fetal bovine serum), may be added to cease digestion activity.

After digestion in chamber 300, the digested cell composition may be passed via valve system 310 to cell concentrator 313, using the force of gravity, suction, or positive pressure such as, in one embodiment, piston 305. When adipose tissue is digested, the digested cell composition will typically comprise at least two phases, including an upper oily phase comprising fat, and a lower aqueous phase comprising a suspension of cells of interest such as adipose stromal cells (ASCs). As the digested cell composition is passed via valve system 310 to cell concentrator 313, the composition may optionally pass a sensor such as, in one embodiment, optical sensor system 323 and 324, which measure a property of the composition, and can sense when the digested cell composition changes. In one embodiment, if the digested cell composition comprises an oily phase, the sensor system can be configured to shut off the flow before the oily phases enters the cell concentrator 313.

In the cell concentrator, wash buffer or cleaning buffer may be passed, in one embodiment, on one side of a membrane, where the other side of the membrane comprises a suspension of the cells of interest. In a preferred embodiment, the cell concentrator 313 may be a hollow-fiber separation module containing capillaries 317 for holding the cell suspension, and in the extracapillary space, a wash buffer or cleaning buffer may be circulated through inlets 318 and 319.

### EXAMPLE 1

In another example, not intended to be limiting, and not intended to represent every aspect of the inventions described herein, lipoaspirate may be mixed with an equal volume of saline, PBS, or media, and may optionally be shaken or stirred (preferably shaken). About 80 mL of the mixture may then be placed into a commercial potato ricer and pressed, thus extruding the non-fibrous content of the sample through the perforations of the ricer while leaving behind the fibrous tissue. If, because of an incomplete seal on the ricer, some of the fat leaks onto the top of the compression disk, it may be recycled to the bottom of the disk and re-pressed. A more preferred compression piston may contain a seal for preventing such leakage.

### EXAMPLE 2

In another example, not intended to be limiting, and not intended to represent every aspect of the inventions described herein, lipoaspirate may be mixed with an equal volume of saline, PBS, or media, and may optionally be shaken or stirred (preferably shaken). 200 mL or more of the mixture may then be passed through a commercial rotary herb mill. The mill may be rotated, and in the process non-fibrous adipose tissue may be passed through the mill, while the fibrous tissue does not pass, and becomes wound up in the blades of the herb mill. During this process, tape may be used to seal off any holes other than the openings between the stationary blades and rotary blades of the herb mill.

### EXAMPLE 3

In another example, not intended to be limiting, and not intended to represent every aspect of the inventions described herein, gauze may be attached to the inside of a commercial salad spinner with lab tape. A suitable salad spinner may, for example, be the OXO Good Grips Little Salad & Herb Spinner provided by OXO International, Ltd. Suitable gauze may, for example, be Venture™ 4 in. by 4 in., 12 ply gauze supplied by TIDI® Products, catalog No. 908272. The gauze may be wetted with approximately 100 mL of a wash buffer and spin in the salad spinner to cause the gauze to stick to the inner bowl of the salad spinner. About 80 mL of lipoaspirant tissue from which fibrous tissue has been removed may be mixed with about 80 mL of wash buffer and placed in the inner bowl of the salad spinner. The mixture may be spun manually or via a motor a sufficient number of times to remove much, preferably most, of the liquid content of the tissue sample. Because of the centrifugal force, the mixture may likely coat the sides of the salad spinner. Fat tissue may then be scraped off the gauze by, for example, a device such as an ice cream scooper.

In the above examples, the components may be automated by connecting the potato ricer, herb grinder, or salad spinner to a motor, and providing seals to ensure the process is carried out aseptically. Tissue samples may be transported between various parts, and from the liposuction cannula, via a syringe, suction, or positive pressure through a tubing, or other equivalent means known in the art.

The above are exemplary modes of carrying out the invention and are not intended to be limiting. It will be apparent to those of ordinary skill in the art that modifications thereto can be made without departure from the spirit and scope of the invention as set forth in the following claims.

What is claimed is:

1. A tissue processing device for extracting cells of interest from an input composition comprising a tissue sample composition and one or more liquid phase compositions, comprising:
first automated means for shredding at least a portion of the input composition, and for separating said portion of the input composition into a fibrous composition and an intermediate cell product composition, wherein the fibrous composition is relatively rich in a fibrous tissue component and the intermediate cell product composition is relatively rich in the cells of interest;
second automated means for separating the one or more liquid phase compositions from the fibrous composition or the intermediate cell product composition, or both; and
an electronic control system capable of starting and stopping the operation of the first automated separating means and for starting and stopping the second automated separating means.

2. The tissue processing device of claim 1, wherein the first automated means comprises:
a solid surface comprising a plurality of perforations large enough to permit passage of at least a portion of the intermediate cell product composition, and small enough to retain the majority of the fibrous composition;
a piston configured to press the input composition against the solid surface with sufficient force to cause at least a portion of the intermediate cell product composition to pass through the perforations.

3. The tissue processing device of claim 1, wherein the first automated means comprises a first plurality of blades configured to sweep against a first set of one or more surfaces in contact with the portion of the input composition.

4. The tissue processing device of claim 3, wherein the first set of one or more surfaces comprises a second plurality of blades configured to be in an offset tooth pattern with the first plurality of blades, and wherein the openings defined by the spaces between the offset teeth are large enough to permit passage of at least a portion of the intermediate cell product composition, and small enough to prevent passage of the majority of the fibrous composition after the first plurality of blades have swept against the second plurality of blades.

5. The tissue processing device of claim 3, wherein the second automated means comprises a second plurality of blades configured to sweep against a second set of one or more surfaces in contact with a composition comprising at least a portion of the intermediate cell product composition.

6. The tissue processing device of claim 5, wherein the second set of one or more surfaces comprises a filter comprising passages or openings large enough to permit passage of the one or more liquid phases, and small enough to retain the majority of the intermediate cell product composition.

7. The tissue processing device of claim 6, wherein the second filter comprises a plurality of beads, wherein each bead is capable of rotating around an axis.

8. The tissue processing device of claim 6, further comprising a basket, wherein the filter forms at least part of the wall of the basket, and the second means further comprises means for rotating the basket to produce a centrifugal force with a vector component normal to at least a portion of the filter.

9. The tissue processing device of claim 8, further comprising collection means for collecting at least a portion of the intermediate cell product composition.

10. The tissue processing device of claim 9, wherein the collection means comprises:
a cavity; and
one or more blades configured to direct at least a portion of the intermediate cell product composition into the cavity as the basket rotates.

11. A method for processing an input composition comprising a tissue sample composition and one or more liquid phase compositions to extract cells of interest, comprising the steps of:
engaging a first automated apparatus for:
shredding at least a portion of the input composition; and
separating said portion of the input composition into a fibrous composition that is relatively rich in a fibrous tissue component and an intermediate cell product composition that is relatively rich in the cells of interest; and
engaging a second automated apparatus to separate the one or more liquid phases from the fibrous composition or the intermediate cell product composition, or both.

12. The method of claim 11, wherein the step of engaging the first automated apparatus has the effect of sweeping a first plurality of blades against a first set of one or more surfaces in contact with at least a portion of the input composition;

13. The method of claim 12, wherein the step of engaging the second automated apparatus has the effect of sweeping a second plurality of blades against a second set of one or more surfaces in contact with a composition comprising at least a portion of the intermediate cell product composition.

14. The method of claim 13,
wherein the second set of one or more surfaces comprises a filter comprising passages or openings large enough to permit passage of the one or more liquid phases, and small enough to retain the majority of the intermediate cell product composition, and
wherein the filter forms at least part of the wall of the basket; and
wherein the step of engaging the second automated apparatus has the further effect of rotating the basket to produce a centrifugal force with a vector component normal to at least a portion of the filter.

15. The method of claim 14, further comprising the step of directing at least a portion of the intermediate cell product composition into a cavity as the basket rotates.

16. A tissue processing device for extracting cells of interest from a first input composition comprising a tissue sample composition and one or more liquid phase compositions, comprising:
a container comprising a first compartment and a second compartment;
first separation means within the first compartment for separating at least a portion of the input composition into a first solid composition that is relatively rich in the tissue sample composition, and a first liquid composition that is relatively rich in the one or more liquid phase compositions;
a gap between the first compartment and the second compartment;
means within the gap for shredding at least a portion of the first solid composition, and for separating said portion of the first solid composition into a fibrous composition and an intermediate cell product composition so as to transfer at least a portion of the intermediate cell product composition to the second compartment while retaining the majority of the fibrous composition outside the second compartment, wherein the fibrous composition is relatively rich in a fibrous tissue component and the intermediate cell product composition is relatively rich in the cells of interest.

17. The tissue processing device of claim 16, further comprising means for rotating the first compartment so that the first solid composition is in contact with the gap.

18. The tissue processing device of claim 16, wherein the first separating means comprises a first plurality of blades configured to sweep against a second plurality of blades in contact with the portion of the first solid composition, wherein the second plurality of blades is configured to be in an offset tooth pattern with the first plurality of blades, and wherein the openings defined by the spaces between the offset teeth are large enough to permit passage of at least a portion of the first solid composition, and small enough to prevent passage of the majority of the fibrous composition after the first plurality of blades have swept against the second plurality of blades.

19. The tissue processing device of claim 16, further comprising second separation means within the second compartment for separating a second input composition into a second solid composition and a second liquid composition,
wherein the second input composition comprises at least a portion of the intermediate cell product composition; and
wherein the second solid composition is relatively rich in the cells of interest.

20. A method for processing an input composition comprising a tissue sample composition and one or more liquid phase compositions to extract cells of interest, comprising the steps of:
injecting the input composition into the first compartment of a container comprising:
a first compartment and a second compartment;
a gap between the first compartment and the second compartment; and
a first plurality of blades within the gap;
separating at least a portion of the input composition into a first solid composition that is relatively rich in the tissue sample composition, and a first liquid composition that is relatively rich in the one or more liquid phase compositions;
sweeping the first plurality of blades against a second plurality of blades in contact with the portion of the first solid composition, wherein the second plurality of blades is configured to be in an offset tooth pattern with the first plurality of blades, and wherein the openings defined by the spaces between the offset teeth are large enough to permit passage of at least a portion of the first solid composition, and small enough to prevent passage of the majority of the fibrous composition after the first plurality of blades have swept against the second plurality of blades; and
separating said portion of the first solid composition into a fibrous composition and an intermediate cell product composition so as to transfer at least a portion of the intermediate cell product composition to the second compartment while retaining the majority of the fibrous composition outside the second compartment, wherein the fibrous composition is relatively rich in a fibrous tissue component and the intermediate cell product composition is relatively rich in the cells of interest.

21. A tissue processing device for extracting cells of interest from a tissue sample composition, comprising:
a digestion chamber comprising a first section and a second section, the first section and the second section separated by a first filter, wherein the first filter is configured to allow passage of particles of a particular size or smaller, while retaining particles of the particular size or larger within the first section of the digestion chamber, wherein the particular size is larger than the cells of interest;
agitation means for agitating a composition within the digestion chamber;
a piston configured within the first section so as to be capable of displacing fluids in either of two directions across the first filter; and
a system of one or more valves configurable in a first configuration to allow fluid communication between the second section and a waste container, configurable in a second configuration to allow fluid communication between the second section and a suspension medium container, and configurable in a third configuration to allow fluid communication with the inlet of a cell concentrator.

22. The tissue processing device of claim 21, wherein the agitation means comprises means for rotating the digestion chamber in one or more directions about an axis.

23. The tissue processing device of claim 21, wherein the cell concentrator is a container comprising:
a first cell concentrator section in fluid communication with the inlet of the cell concentrator, separated from a second cell concentrator section by a second filter, wherein the second filter is configured to allow passage of liquids, while retaining particles at least the size of the cells of interest;
a cell outlet configurable to be in fluid communication with the first cell concentrator section;
a wash inlet configurable to be in fluid communication with the second cell concentrator section; and
a wash outlet configurable to be in fluid communication with the second cell concentrator section.

24. The tissue processing device of claim 23, wherein the first filter comprises a mesh, and the second filter comprises a plurality of hollow fibers.

25. The tissue processing device of claim 21, further comprising:
a passage between the one or more valves and the input to the cell concentrator;
a sensor configured to measure one or more physical or chemical properties of the contents of the passage; and

26. The tissue processing device of claim 25, further comprising a control system connected to the sensor configured to be capable of controlling the movement of the piston.

27. The tissue processing device of claim 21, further comprising:
first automated means for shredding at least a portion of an input composition comprising the tissue sample composition and one or more liquid phase compositions, and for separating said portion of the input composition into a fibrous composition and an intermediate cell product composition, wherein the fibrous composition is relatively rich in a fibrous tissue component and the intermediate cell product composition is relatively rich in the cells of interest;
second automated means for separating the one or more liquid phase compositions from the fibrous composition or the intermediate cell product composition, or both;
an electronic control system capable of starting and stopping the operation of the first automated separating means and for starting and stopping the second automated separating means;
collection means for collecting at least a portion of the intermediate cell product composition; and
transport means for transporting at least a portion of the intermediate cell product composition to an inlet of the first section of the digestion chamber.

28. The tissue processing device of claim 21, further comprising:
a container comprising a first compartment and a second compartment;
first separation means within the first compartment for separating at least a portion of an input composition into a first solid composition that is relatively rich in the tissue sample composition, and a first liquid composition that is relatively rich in the one or more liquid phase compositions, wherein the input composition comprises the tissue sample composition and one or more liquid phase compositions;
a gap between the first compartment and the second compartment;
means within the gap for shredding at least a portion of the first solid composition, and for separating said portion of the first solid composition into a fibrous composition and an intermediate cell product composition so as to transfer at least a portion of the intermediate cell product composition to the second compartment while retaining the majority of the fibrous composition outside the second compartment, wherein the fibrous composition is relatively rich in a fibrous tissue component and the intermediate cell product composition is relatively rich in the cells of interest;
collection means for collecting at least a portion of the intermediate cell product composition; and
transport means for transporting at least a portion of the intermediate cell product composition to an inlet of the first section of the digestion chamber.

29. A method for processing a tissue sample composition to extract cells of interest, comprising the steps of:
introducing the tissue sample composition into a digestion chamber comprising a first section and a second section, the first section and the second section separated by a first filter, wherein the first filter is configured to allow passage of particles of a particular size or smaller, while retaining particles of the particular size or larger within the first section of the digestion chamber, wherein the particular size is larger than the cells of interest;
introducing a suspension medium into the first section of the digestion chamber;
allowing the contents of the first section of the digestion chamber to digest for a predetermined length of time, to create a digested cell composition;
pushing or suctioning at least a portion of the digested cell composition through the first filter;
passing said portion of the digested cell composition through a passage toward the inlet of a cell concentrator; and
passing at least a part of said portion of the digested cell composition through the cell concentrator.

30. The method of claim 29, further comprising the steps of:
introducing a wash buffer into the first section after the tissue sample composition has been introduced into the digestion chamber;
agitating the contents of the first section; and
pushing, suctioning, or draining the wash buffer through the first filter.

31. The method of claim 29, wherein said portion of the digested cell composition comprises an oil phase and an aqueous cell suspension phase, further comprising the step of monitoring a physical or chemical property of said portion of the digested cell composition with a sensor at a sensor location, wherein the sensor is capable of generating a signal indicating whether the digested cell composition at the sensor location comprises the oil phase or the aqueous cell suspension phase.

32. The method of claim 29, further comprising the steps of:
concentrating said part of said portion of the digested cell composition within the cell concentrator; and
collecting a composition containing the cells of interest from the cell concentrator.

33. The method of claim 32, wherein the step of concentrating further comprises the steps of:
situating said part of said portion of the digested cell composition on one side of one or more filter membranes with pores smaller than the diameter;
providing conditions whereby a fluid from the digested cell composition passes across the one or more filter membranes.
